(19) United States
(12) Patent Application Publication
(10) Pub. No.: US 2025/0213638 A1
(43) Pub. Date: Jul. 3, 2025

(54) Topical Pain Cream

(71) Applicant: MG Science Technologies LLC, Delray Beach, FL (US)

(72) Inventor: Michael R. Anderson, Delray Beach, FL (US)

(73) Assignee: MG Science Technologies LLC, Delray Beach, FL (US)

(21) Appl. No.: 19/003,129

(22) Filed: Dec. 27, 2024

Related U.S. Application Data
(60) Provisional application No. 63/616,229, filed on Dec. 29, 2023.

(51) Int. Cl. A61K 36/81; A61K 31/00; A61K 31/015; A61K 31/05; A61K 31/10; A61K 31/198; A61K 35/12; A61K 36/28; A61K 36/53; A61K 36/534; A61K 36/54; A61K 36/61; A61K 36/752; A61K 36/8965; A61K 36/9066; A61K 47/32; A61K 47/42

(52) U.S. Cl. CPC A61K 36/81; A61K 31/015; A61K 31/05; A61K 31/10; A61K 31/198; A61K 31/658; A61K 35/12; A61K 36/28; A61K 36/53; A61K 36/534; A61K 36/54; A61K 36/61; A61K 36/752; A61K 36/8965; A61K 36/9066; A61K 47/32; A61K 47/42

(57) ABSTRACT

Pain relief compositions are described for topical application to penetrate deep into the skin, muscles, and joints. The pain relief compositions are prepared as creams for topical applying to the skin. The pain relief compositions contain active ingredients such as DMSO, capsaicin, menthol, eucalyptus extract, chamomile oil, methyl salicylate, and cayenne oil. One or more of the active ingredients is included in the pain relief composition as nanoparticles. Methods for preparing and for using the pain relief compositions are also described.

### CROSS-REFERENCE TO RELATED APPLICATIONS

This application is a nonprovisional application of and claims priority from U.S. provisional patent application Ser. No. 63/616,229 filed on Dec. 29, 2023. The foregoing application is incorporated in its entirety herein by reference.

### FIELD OF THE INVENTION

The invention relates to pain relief compositions, and more particularly, to pain relief creams that can be applied topically for local relief of pain and inflammation in muscles, nerves, bones, and joints, caused by injury or age-related pain.

### BACKGROUND

Topically applied compositions have been used for pain relief of muscles and joints, including pain caused by arthritis and inflammation resulting from injuries suffered during accidents and in sports. Many conventional topically applied pain relief products only provide a cold or hot sensation that is believed to interfere with a pain signal to the brain that in some cases can provide the user a perception of experiencing less pain. However, such conventional pain relief products treat only the skin surface and are incapable of penetrating deeper into the skin, muscles, and joints to relieve pain.

Many topical pain relief products also are incapable of providing both rapid, immediate relief from pain and discomfort in the skin, muscles, joints, and other tissues as well as gradual, longer lasting pain relief such as a time-release or extended-release effect.

A need exists for topical pain relief compositions that are capable of penetrating through the skin surface deeper into a user's skin, muscles, joints, and other tissues to relieve pain and discomfort more effectively than standard topical pain relief compositions.

A need also exists for topical pain relief compositions that can provide both rapid, immediate relief from pain and discomfort in the skin, muscles, joints, and other tissues as well as gradual, longer lasting pain relief such as or similar to a time-release or extended-release effect.

### SUMMARY

This invention relates to pain relief compositions including compositions for topical application. Some embodiments of the compositions provide both heat and cold sensations. The pain relief compositions have been made using nanotechnology to produce nanoparticles of one or more of the active ingredients to allow the active ingredients to penetrate deep into the skin and into muscles and joints to help treat areas of the body experiencing pain rather than just treating the skin surface. Testing has demonstrated that the initial feel or sensation caused by menthol, black pepper, and capsaicin can greatly reduce or eliminate pain to the areas of the skin where the pain relief composition is topically applied. The pain relief compositions contain active ingredients such as DMSO, capsaicin, menthol, eucalyptus extract, chamomile oil, methyl salicylate, and cayenne oil. The active ingredients of the pain relief compositions described herein can substantially reduce or even eliminate pain and discomfort that a human or animal feels when applied topically to a body part in which pain is being experiencing.

In preparing the pain relief compositions described herein, techniques are used to prepare some of the active ingredients such as shear mixing and nanosizing certain percentages of ingredients for better penetration into the skin, muscle, joints, and other tissues for quicker and longer lasting results.

The pain relief compositions described herein provide an advantage over standard pain relief products by penetrating through the skin surface deeper into a user's skin, muscles, and joints to relieve pain more effectively. The pain relief compositions described herein provide another advantage in more effectively treating skin, muscle, and joint pain by harnessing the ability of nanoparticles of active ingredients to penetrate through the skin surface and into the body in ways of which standard topical pain relief compositions are incapable. The pain relief compositions described herein have yet another advantage in providing both rapid, immediate relief from pain and discomfort in the skin, muscles, joints, and other tissues as well as gradual, longer lasting pain relief such as or similar to a time-release or extended-release effect.

Accordingly, the invention features a pain relief composition that includes effective amounts of the following ingredients: one or more extracts from a plant of the genus Capsicum; menthol; cayenne oil; dimethylsulfoxide (“DMSO”); tea tree oil; emu oil; turmeric oil; peppermint oil; avocado oil; lavender oil; chamomile oil; eucalyptus extract; methylsulfonylmethane (“MSM”); spearmint extract; one or more salts of ethylenediaminetetraacetic acid (“EDTA”); sodium acid sulphate; methyl salicylate; aloe vera; camphor oil; lemon extract; and extract of Arnica montana.

In another aspect, the invention can feature the one or more extracts from a plant of the genus Capsicum being or including capsaicin.

In another aspect, the invention can feature the one or more cannabinoids being or including hemp seed oil, cannabidiol (“CBD”), or both.

In another aspect, the invention can feature at least one of the ingredients being or including nanoparticles of such ingredient.

In another aspect, the invention can further include carbomer 980 or another thickening agent.

In another aspect, the invention can further include water.

In another aspect, the invention can feature one or more preservatives selected from among the following group: nisin; phenoxyethanol; methylparaben; propylparaben; butylparaben; any other paraben safe for use as a preservative; sodium benzoate; potassium sorbate; benzyl alcohol; chlorphenesin; diazolidinyl urea; imidazolidinyl urea; sorbic acid; caprylyl glycol; dehydroacetic acid; tetrasodium ethylenediaminetetraacetic acid (“tetrasodium EDTA”); sodium dehydroacetate; benzoic acid; methylisothiazolinone (“MIT”); and ethylhexylglycerin.

In another aspect, the invention can further include nisin as a preservative.

The invention also features a pain relief composition that includes effective amounts of the following ingredients: one or more extracts from a plant of the genus Capsicum; menthol; cayenne oil; dimethylsulfoxide (“DMSO”); tea tree oil; emu oil; turmeric oil; peppermint oil; avocado oil; lavender oil; chamomile oil; eucalyptus extract; methylsulfonylmethane (“MSM”); spearmint extract; one or more salts of ethylenediaminetetraacetic acid (“EDTA”); sodium acid sulphate; methyl salicylate; aloe vera; camphor oil; lemon extract; and extract of Arnica montana. At least one of the foregoing ingredients is or includes nanoparticles of such ingredient. The pain relief composition further includes carbomer 980, one or more preservatives, and water.

A method of the invention can be used to prepare a pain relief composition. The method includes the steps of: (a) heating and liquefying menthol; (b) adding methyl salicylate and dimethylsulfoxide (“DMSO”) to the liquefied menthol with mixing to create a first mixture; (c) adding the following ingredients to the first mixture: one or more extracts from a plant of the genus Capsicum; cayenne oil; tea tree oil; emu oil; turmeric oil; peppermint oil; avocado oil; lavender oil; chamomile oil; eucalyptus extract; spearmint extract; one or more salts of ethylenediaminetetraacetic acid (“EDTA”); aloe vera; camphor oil; lemon extract; and extract of Arnica montana; (d) mixing methylsulfonylmethane (“MSM”) and sodium acid sulphate to create a second mixture; and (e) mixing the first mixture and the second mixture to create a pain relief composition.

Another method of the invention can include step (a) further including heating the menthol to a temperature of about 50° C. or more.

Another method of the invention can include at least one ingredient identified in steps (a), (b), (c), and (d) being or including nanoparticles of such ingredient.

Another method of the invention can include the step of: (f) adding and mixing carbomer 980 to the mixture created in step (e).

Another method of the invention can include step (e) being performed for about 15 minutes.

Another method of the invention can include step (e) being performed using a high shear mixer.

Another method of the invention can include step (f) being performed about 10 minutes after step (e).

Another method of the invention can include steps (a), (b), and (c) being performed in a first container and step (d) being performed in a second container.

Another method of the invention can include one or more of the following additional ingredients also being added to the first mixture in step (c): hemp seed oil and cannabidiol (“CBD”).

Another method of the invention can include, in at least one of steps (c), (d), or (e), one or more preservatives being added by mixing, wherein the one or more preservatives are selected from among the following group: nisin; phenoxyethanol; methylparaben; propylparaben; butylparaben; any other paraben safe for use as a preservative; sodium benzoate; potassium sorbate; benzyl alcohol; chlorphenesin; diazolidinyl urea; imidazolidinyl urea; sorbic acid; caprylyl glycol; dehydroacetic acid; tetrasodium ethylenediaminetetraacetic acid (“tetrasodium EDTA”); sodium dehydroacetate; benzoic acid; methylisothiazolinone (“MIT”); and ethylhexylglycerin.

Another method of the invention can include, in at least one of steps (c), (d), or (e), one or more preservatives including at least nisin being added by mixing.

Unless otherwise defined, all technical terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents and other references mentioned herein are incorporated by reference in their entirety. In the case of conflict, the present specification, including definitions will control.

### DETAILED DESCRIPTION

The present invention is best understood by reference to the description set forth herein. Embodiments of the invention are discussed below; however, those skilled in the art will readily appreciate that the detailed description given herein is for explanatory purposes as the invention extends beyond these limited embodiments. For example, in light of the teachings of the present invention, those skilled in the art will recognize a multiplicity of alternate and suitable approaches, depending upon the needs of the particular application, to implement the functionality of any given detail described herein beyond the particular implementation choices in the following embodiments described and shown. That is, numerous modifications and variations of the invention may exist that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

The present invention should not be limited to the particular methodology, compounds, materials, manufacturing techniques, uses, and applications, described herein, as these may vary. The terminology used herein is used for the purpose of describing particular embodiments only, and is not intended to limit the scope of the present invention. As used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” may be a reference to one or more steps or means and may include sub-steps and subservient means.

All conjunctions used herein are to be understood in the most inclusive sense possible. Thus, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should be read as “and/or” unless expressly stated otherwise. Structures described herein are to be understood also to refer to functional equivalents of such structures. Language that may be construed to express approximation should be so understood unless the context clearly dictates otherwise.

Unless otherwise defined, all terms (including technical and scientific terms) are to be given their ordinary and customary meaning to a person of ordinary skill in the art, and are not to be limited to a special or customized meaning unless expressly so defined herein.

Terms and phrases used in this application, and variations thereof, especially in the appended claims, unless otherwise expressly stated, should be construed as open ended as opposed to limiting. As examples of the foregoing, the term “including” should be read to mean “including, without limitation,” “including but not limited to,” or the like; the term “having” should be interpreted as “having at least”; the term “includes” should be interpreted as “includes but is not limited to”; the term “example” is used to provide exemplary instances of the item in discussion, not an exhaustive or limiting list thereof; and use of terms like “preferably,” “preferred,” “desired,” “desirable,” or “exemplary” and words of similar meaning should not be understood as implying that certain features are critical, essential, or even important to the structure or function of the invention, but instead as merely intended to highlight alternative or additional features that may or may not be utilized in a particular embodiment of the invention.

Those skilled in the art will also understand that if a specific number of an introduced claim recitation is intended, such an intent will be explicitly recited in the claim, and in the absence of such recitation no such intent is present. For example, as an aid to understanding, the appended claims may contain usage of the introductory phrases “at least one” and “one or more” to introduce claim recitations; however, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C” is used, in general, such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.).

All numbers expressing dimensions, quantities of ingredients, reaction conditions, and so forth used in the specification are to be understood as being modified in all instances by the term “about” unless expressly stated otherwise. Accordingly, unless indicated to the contrary, the numerical parameters set forth herein are approximations that may vary depending upon the desired properties sought to be obtained.

The invention provides a composition for relieving, reducing, or eliminating pain in the skin, muscles, joints, and other tissues. The pain relief composition is produced as a cream or a lotion for topical application to human skin. In other embodiments, the pain relief composition may be applied to skin of an animal. Active ingredients of the pain relief composition help to relieve, reduce, or eliminate pain and inflammation, including pain and inflammation caused by injury. The pain relief composition includes active ingredients that are formulated to provide relief from nerve, bruise, and arthritis-related pain and discomfort. The cooling and heat sensations that the pain relief composition causes help to promote faster healing. The pain relief composition is very effective at reducing or eliminating muscle pain and discomfort in the upper and lower back and all joints. The pain relief composition may be topically applied in conjunction with and by massaging of the affected areas to provide rapid pain relief generally within minutes. The pain relief composition effectively treats pain and discomfort resulting from arthritis, nerve injuries and conditions, tendonitis, accidents, and sports-related injuries.

The pain relief composition has a spreadable type of texture, which when applied to the skin of a human or animal, can penetrate the skin and past the skin layers. The pain relief composition's consistency, among its several embodiments, may vary based on the combination of the ingredients, oils, water, and emulsifiers, which are absorbed into the body through the skin. Formulations are differentiated by the amount of active ingredients, with some being therapeutically recognized as pain relief active ingredients. The pain relief composition may be produced based on a formula that is considered to be a maximum strength product for more severe pain.

Some of the ingredients have been “nanosized” into nanoparticles for quicker absorption for maximum benefit to the human or animal in delivering rapid and long-lasting pain relief. Many ingredients of conventional pain relief compositions that are not provided as nanoparticles cannot be absorbed past the body's skin layers. Incorporating at least one or more of the active ingredients as nanoparticles of varying sizes provides great benefits related to skin penetration to more effectively and quickly reach muscles, tendons, and other tissues beneath the skin, thereby reducing or eliminating pain and discomfort by reducing inflammation and targeting pain receptor signals at the source of the pain.

As used herein, the term “nanoparticles” means particles of compounds ranging in size from about 1 or less nanometers (nm) to about 100 or more nanometers in diameter or thickness. Nanoparticles of about 1 or less nanometers to about 10 nanometers may be referred to herein as “small nanoparticles.” Nanoparticles of greater than about 10 nanometers to about 50 nanometers may be referred to herein as “medium-sized nanoparticles.” Nanoparticles of greater than about 50 nanometers to about 100 or more nanometers may be referred to herein as “large nanoparticles.”

After topical application of the pain relief composition to a user's skin, small nanoparticles of active ingredients pass quickly and easily through the skin's surface and upper dermal layers to provide immediate relief from pain and discomfort by reducing inflammation and targeting pain receptor signals at the source of the pain.

After topical application of the pain relief composition to a user's skin, medium-sized nanoparticles of active ingredients pass quickly and easily through the skin's surface and upper dermal layers to reach tissues beneath the skin such as tendons and ligaments. The medium-sized nanoparticles of active ingredients allow for deeper absorption of the pain relief composition for sustained pain relief and anti-inflammatory effects at the source of the pain.

After topical application of the pain relief composition to a user's skin, large nanoparticles of active ingredients are absorbed more gradually through the skin's surface and upper dermal layers, which provides a time-release effect since the large nanoparticles reach subcutaneous tissues more slowly than do the small and medium-sized nanoparticles. The large nanoparticles of active ingredients also allow for pain relief in deeper muscle, joints, and other tissues to be extended over several hours to provide longer lasting pain relief.

The use of nanoparticles of varying sizes as described above provides better, more effective pain relief by a dual-action mechanism of quick penetration through the skin for immediate pain relief and slower, more gradual penetration into deeper tissues beneath the skin for prolonged pain relief action to provide extended comfort. The pain relief composition is able to treat both acute and chronic pain due to its utilization of this nanotechnology.

In one exemplary embodiment, active ingredients of the pain relief composition include effective amounts of the following: one or more extracts from a plant of the genus Capsicum; menthol; cayenne oil; dimethylsulfoxide (“DMSO”); tea tree oil; emu oil; turmeric oil; peppermint oil; avocado oil; lavender oil; chamomile oil; eucalyptus extract; methylsulfonylmethane (“MSM”); spearmint extract; one or more salts of ethylenediaminetetraacetic acid (“EDTA”); sodium acid sulphate; methyl salicylate; aloe vera; camphor oil; lemon extract; and extract of Arnica montana. At least one of the active ingredients includes nanoparticles of such active ingredient, and the nanoparticles of such ingredient may all be of generally the same size (e.g., small nanoparticles) or they may be more than one size (e.g., both small and medium-sized, small and large, medium-sized and large, or a combination of all three sizes of nanoparticles). In exemplary embodiments, the one or more extracts from a plant of the genus Capsicum include capsaicin. In other embodiments, one or more of the foregoing active ingredients may be omitted from the pain relief composition. For example, in one embodiment, the pain relief composition may include only the following active ingredients: capsaicin, menthol, DMSO, eucalyptus extract, chamomile oil, methyl salicylate, and cayenne oil.

In exemplary embodiments, the composition also includes a thickening agent, one or more preservatives, and water. The thickening agent can be carbomer 980 or another thickening agent.

Suitable preservatives for inclusion in the pain relief composition include one or more preservatives selected from among: nisin; phenoxyethanol; methylparaben; propylparaben; butylparaben; any other paraben safe for use as a preservative; sodium benzoate; potassium sorbate; benzyl alcohol; chlorphenesin; diazolidinyl urea; imidazolidinyl urea; sorbic acid; caprylyl glycol; dehydroacetic acid; tetrasodium ethylenediaminetetraacetic acid (“tetrasodium EDTA”); sodium dehydroacetate; benzoic acid; methylisothiazolinone (“MIT”); and ethylhexylglycerin. In exemplary embodiments of the pain relief composition, nisin is the preservative.

In some embodiments, the pain relief composition also includes one or more cannabinoids, including sources of cannabinoids. The cannabinoids can be selected from among hemp seed oil, cannabidiol (“CBD”), or both. Any other suitable cannabinoid that provides pain relief effects may also be included in various embodiments of the pain relief composition.

In one exemplary embodiment, the pain relief composition includes effective amounts of the following active ingredients in addition to carbomer 980, one or more preservatives, and water:
one or more extracts from a plant of the genus Capsicum
menthol
cayenne oil
DMSO
tea tree oil
emu oil
turmeric oil
peppermint oil
avocado oil
lavender oil
chamomile oil
eucalyptus extract
MSM
spearmint extract
one or more salts of EDTA
sodium acid sulphate
methyl salicylate
aloe vera
camphor oil
lemon extract
extract of Arnica montana

In this exemplary embodiment, at least one of the foregoing active ingredients includes nanoparticles of such ingredient.

In a more specific example of the preceding exemplary embodiment, the pain relief composition includes effective amounts of the following active ingredients in addition to carbomer 980, one or more preservatives, and water:
about 0.01% to about 5.50% capsaicin
about 1% to about 10.9% menthol
about 0.2% to about 8.5% cayenne oil
about 2.5% to about 83% DMSO
about 0.12% to about 5.3% tea tree oil
about 0.015% to about 4.5% emu oil
about 0.11% to about 5.3% turmeric oil
about 0.1% to about 4.5% peppermint oil
about 0.13% to about 5.5% avocado oil
about 0.010% to about 3.7% lavender oil
about 0.015% to about 4.5% chamomile oil
about 0.10% to about 7.5% eucalyptus extract
about 0.10% to about 5.5% MSM
about 0.11% to about 4.9% spearmint extract
about 0.01% to about 6.2% one or more salts of EDTA
about 0.002% to about 3.2% sodium acid sulphate
about 0.30% to about 45.5% methyl salicylate
about 0.10% to about 14.5% aloe vera
about 0.10% to about 6.5% camphor oil
lemon extract
about 0.002% to about 3.1% extract of Arnica montana

As mentioned above, in this exemplary embodiment, at least one of the foregoing active ingredients includes nanoparticles of such ingredient.

The invention also includes a method for preparing or producing the pain relief compositions described herein. The method includes the step of heating and liquefying menthol (also referred to herein as “step (a)”). The menthol may be or may include nanoparticles of menthol. The menthol is heated in a first container to a temperature of about 50° C. or more.

In a next step of the method (also referred to herein as “step (b)”), methyl salicylate and dimethylsulfoxide (“DMSO”) are added to the liquefied menthol with mixing to create a first mixture. At least one of the foregoing ingredients identified in this step may be or may include nanoparticles of such ingredient. This step of the method may also be performed in the first container or in a second container.

In a next step of the method (also referred to herein as “step (c)”), the following ingredients are added to the first mixture: one or more extracts from a plant of the genus Capsicum; cayenne oil; tea tree oil; emu oil; turmeric oil; peppermint oil; avocado oil; lavender oil; chamomile oil; eucalyptus extract; spearmint extract; one or more salts of ethylenediaminetetraacetic acid (“EDTA”); aloe vera; camphor oil; lemon extract; and extract of Arnica montana. At least one of the foregoing ingredients identified in this step may be or may include nanoparticles of such ingredient. This step of the method may also be performed in the first container or in a second or other container.

In a next step of the method (also referred to herein as “step (d)”), methylsulfonylmethane (“MSM”) and sodium acid sulphate are mixed to create a second mixture. At least one ingredient identified in this step may be or may include nanoparticles of such ingredient. This step of the method may also be performed in the first container or in a second or other container.

Finally, in a next step of the method (also referred to herein as “step (e)”), the first mixture and the second mixture are mixed to create a pain relief composition. This mixing is performed for about 15 minutes. A high shear mixer may be used to perform this mixing. This step of the method is performed in a second container or in the first or another container.

In some embodiments, the method may also include the step of adding and mixing carbomer 980 to the mixture created when the first mixture and the second mixture are mixed to create the pain relief composition. This step of adding and mixing in carbomer 980 (also referred to herein as “step (f)”) is performed about 10 minutes after the first mixture and the second mixture are mixed to create the pain relief composition.

In some embodiments of the method, during step (d), hemp seed oil, cannabidiol (“CBD”), or both may also be added to the first mixture.

In exemplary embodiments of the method, during at least one of steps (c), (d), or (e), one or more preservatives are added by mixing. The one or more preservatives are selected from among: nisin; phenoxyethanol; methylparaben; propylparaben; butylparaben; any other paraben safe for use as a preservative; sodium benzoate; potassium sorbate; benzyl alcohol; chlorphenesin; diazolidinyl urea; imidazolidinyl urea; sorbic acid; caprylyl glycol; dehydroacetic acid; tetrasodium ethylenediaminetetraacetic acid (“tetrasodium EDTA”); sodium dehydroacetate; benzoic acid; methylisothiazolinone (“MIT”); and ethylhexylglycerin. In the most exemplary embodiments, the one or more preservatives includes nisin.

As an example of one embodiment of a method for preparing a pain relief composition as described herein, menthol is heated in a first container to a temperature of about 50° C. or more until liquefied. Next, and after discontinuing heating of the liquefied menthol, methyl salicylate and DMSO are added to liquefied menthol and dispersed well to create a first mixture. Next, after the menthol, methyl salicylate, and DMSO are very well blended, the following are added in a separate container and mixed very quickly to reduce the recrystallization of menthol with mixing, thereby creating a second mixture: one or more extracts from a plant of the genus Capsicum; cayenne oil; tea tree oil; emu oil; turmeric oil; peppermint oil; avocado oil; lavender oil; chamomile oil; eucalyptus extract; spearmint extract; one or more salts of ethylenediaminetetraacetic acid (“EDTA”); aloe vera; camphor oil; lemon extract; and extract of Arnica montana. After the second mixture is prepared, it is blended for approximately 15 minutes using a high shear mixer. Next, carbomer 980 is added and mixed for about 10 minutes to produce a pain relief composition in the form of a cream having a uniform consistency. At the step in which the second mixture is created, one or more cannabinoids or one or more sources of cannabinoids may be added such as, for example, hemp seed oil, cannabidiol (“CBD”), or any other suitable cannabinoid.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A pain relief composition comprising effective amounts of the following ingredients:
one or more extracts from a plant of the genus Capsicum;
menthol;
cayenne oil;
dimethylsulfoxide (“DMSO”);
tea tree oil;
emu oil;
turmeric oil;
peppermint oil;
avocado oil;
lavender oil;
chamomile oil;
eucalyptus extract;
methylsulfonylmethane (“MSM”);
spearmint extract;
one or more salts of ethylenediaminetetraacetic acid (“EDTA”);
sodium acid sulphate;
methyl salicylate;
aloe vera;
camphor oil;
lemon extract; and
extract of Arnica montana.

2. The composition of claim 1, wherein the one or more extracts from a plant of the genus Capsicum comprise capsaicin.

3. The composition of claim 1, further comprising one or more cannabinoids comprising hemp seed oil, cannabidiol (“CBD”), or both.

4. The composition of claim 1, wherein at least one of the ingredients is comprised of nanoparticles of such ingredient.

5. The composition of claim 1, further comprising carbomer 980 or another thickening agent.

6. The composition of claim 1, further comprising water.

7. The composition of claim 1, further comprising one or more preservatives selected from among the group consisting of: nisin; phenoxyethanol; methylparaben; propylparaben; butylparaben; any other paraben safe for use as a preservative; sodium benzoate; potassium sorbate; benzyl alcohol; chlorphenesin; diazolidinyl urea; imidazolidinyl urea; sorbic acid; caprylyl glycol; dehydroacetic acid; tetrasodium ethylenediaminetetraacetic acid (“tetrasodium EDTA”); sodium dehydroacetate; benzoic acid; methylisothiazolinone (“MIT”); and ethylhexylglycerin.

8. The composition of claim 1, further comprising nisin as a preservative.

9. A pain relief composition comprising effective amounts of the following ingredients:
one or more extracts from a plant of the genus Capsicum;
menthol;
cayenne oil;
dimethylsulfoxide (“DMSO”);
tea tree oil;
emu oil;
turmeric oil;
peppermint oil;
avocado oil;
lavender oil;
chamomile oil;
eucalyptus extract;
methylsulfonylmethane (“MSM”);
spearmint extract;
one or more salts of ethylenediaminetetraacetic acid (“EDTA”);
sodium acid sulphate;
methyl salicylate;
aloe vera;
camphor oil;
lemon extract; and
extract of Arnica montana;
wherein at least one of the foregoing ingredients is comprised of nanoparticles of such ingredient; and
further comprising: carbomer 980; one or more preservatives; and water.

10. A method for preparing a pain relief composition, the method comprising the steps of:
(a) heating and liquefying menthol;
(b) adding methyl salicylate and dimethylsulfoxide (“DMSO”) to the liquefied menthol with mixing to create a first mixture;
(c) adding the following ingredients to the first mixture: one or more extracts from a plant of the genus Capsicum; cayenne oil; tea tree oil; emu oil; turmeric oil; peppermint oil;
avocado oil; lavender oil; chamomile oil; eucalyptus extract; spearmint extract; one or more salts of ethylenediaminetetraacetic acid (“EDTA”); aloe vera; camphor oil; lemon extract; and extract of Arnica montana;
(d) mixing methylsulfonylmethane (“MSM”) and sodium acid sulphate to create a second mixture; and
(e) mixing the first mixture and the second mixture to create a pain relief composition.

11. The method of claim 10, wherein step (a) comprises heating the menthol to a temperature of about 50° C. or more.

12. The method of claim 10, wherein at least one ingredient identified in steps (a), (b), (c), and (d) is comprised of nanoparticles of such ingredient.

13. The method of claim 10, further comprising the step of:
(f) adding and mixing carbomer 980 to the mixture created in step (e).

14. The method of claim 10, wherein step (e) is performed for about 15 minutes.

15. The method of claim 10, wherein step (e) is performed using a high shear mixer.

16. The method of claim 13, wherein step (f) is performed about 10 minutes after step (e).

17. The method of claim 10, wherein steps (a), (b), and (c) are performed in a first container and step (d) is performed in a second container.

18. The method of claim 10, wherein one or more of the following additional ingredients are also added to the first mixture in step (c): hemp seed oil and cannabidiol (“CBD”).

19. The method of claim 10, wherein in at least one of steps (c), (d), or (e), one or more preservatives are added by mixing, wherein the one or more preservatives are selected from among the group consisting of: nisin; phenoxyethanol; methylparaben; propylparaben; butylparaben; any other paraben safe for use as a preservative; sodium benzoate; potassium sorbate; benzyl alcohol; chlorphenesin; diazolidinyl urea; imidazolidinyl urea; sorbic acid; caprylyl glycol; dehydroacetic acid; tetrasodium ethylenediaminetetraacetic acid (“tetrasodium EDTA”); sodium dehydroacetate; benzoic acid; methylisothiazolinone (“MIT”); and ethylhexylglycerin.

20. The method of claim 10, wherein in at least one of steps (c), (d), or (e), one or more preservatives comprising at least nisin are added by mixing.
